(19) United States
(12) Patent Application Publication
(10) Pub. No.: US 2006/0257469 A1
(43) Pub. Date: Nov. 16, 2006

(54) Enhanced indoleamine and catecholamine bio-availability via catechin inhibition of L-Dopa decarboxylase

(76) Inventor: Yochanan R. Bulka, Lakewood, NJ (US)

Correspondence Address: LIFE SCIENCE LABORATORIES, INC., 170 N. OBERLIN AVE, UNIT 26, LAKEWOOD, NJ, 08701, US

(21) Appl. No.: 11/398,252

(22) Filed: Apr. 5, 2006

Related U.S. Application Data
(60) Provisional application No. 60/594,406, filed on Apr. 5, 2005.

(51) Int. Cl. A61K 31/405; A61K 31/353; A61K 9/48; A61K 9/20

(52) U.S. Cl. 424/451; 514/419; 514/456; 424/464

(57) ABSTRACT

It is the embodiment of this invention to form novel compositions of Indoleamines e.g. 5-Hydroxytryptophan (5HTP) and/or Catecholamines e.g. L-Dopa with the gallocatechins e.g. (−)epigallocatechin3-O-gallate (EGCG) and/or (−) epigallocatechin (EGC) or any of the catechins found in green tea in a pharmaceutical or nutritional dosage or dietary regimen be it in tablet, liquid, capsule, injectable or any other ingestible form to achieve enhanced bioavailability and a superior safety profile.

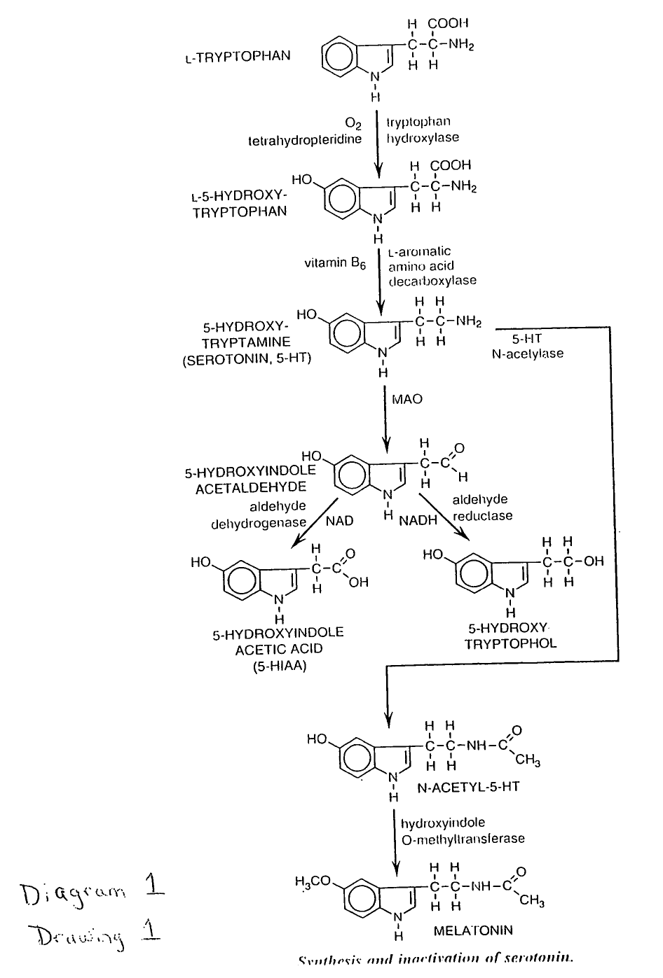

Supplementation of Serotonin (5-HT/5-Hydroxytryptamine) a key neurotransmitter into the Central Nervous System (CNS), has proved to be effective against many medical conditions, including but not limited to depression, obesity, fibromyalgia, myoclonus and others. 5-Hydroxytryptophan is decarboxylated to Serotonin via the ubiquitous enzyme L-Dopa Decarboxylase. To avoid premature decarboxylation several drugs i.e. Carbidopa and Benserazide have been employed as inhibitors of this enzyme. This is needed to ensure that the decarboxylation process occurs primarily after 5-HTP crosses the blood-brain barrier. Therefore, there will be greater safety and efficacy by minimizing the amount of circulatory Serotonin, which can have undesirable side-effects, and increasing the amount of Serotonin available in the CNS.

In a similar fashion Levodopa effectively used for treatment of Parkinson's disease is combined with Carbidopa and/or Benserazide for similar purposes.

We have been able to achieve the same effects using the non-pharmaceutical gallocatechins in place of Carbidopa and Benserazide. Surprisingly, in clinical trials we have achieved similar and in some cases superior results as measured by clinical markers and blood and urinary metabolites

### DETAILED DESCRIPTION

As a Supplement to the Brief Description Above We Have as Follows:

This invention relates to a novel method for inhibiting the ubiquitious enzyme Aromatic Amino Acid Decarboxylase hereinafter referred to as (AADC) via non-pharmaceutical moieties

The applications of said invention span a broad range, including but not limited to depression, Parkinson's disease, weight loss etc,

The discussion and examples set forth herein with regard to 5-Hydroxytryptophan (5-HTP) can be analogously used with regard to L-Dopa

The conversion of (5-HTP) to serotonin via (AADC) is shown in Diagram 1 To avoid its conversion to Serotonin in the periphery several pharmaceutical (s) have been found which in combination inhibit (AADC) so as to allow Serotonin conversion in the Central Nervous System (CNS) i.e. Carbidopa and Benserazide

The galloatachins referred to in the abstract (−) epigallocatechin-3-O-gallate (EGCG) and/or (−) epigallocatechin (EGC) have such properties.

Surprisingly in the two clinicals (Example 1 and 2 below) the combination showed superiority to the Pharmaceutical combinations in these respects
1. Lower Serotonin content in the circulatory system
2. Less side affects
3. Lower measured amounts of the key negative metabolic indicator 5-hydroxyindole acetic acid
4. Indications of a preferable metabolic time-profile consistent with the desired peripheral decarboxylation

Example 1—10 subjects were given a tablet twice a day for five days containing 50 mg (5-HTP), 90 mg (EGCG)/(ECGC), 100 mg Tyrosine ang 100 mg Phenylalanine

Example 2—Same as 1 but duration of dosaging was for 15 days!

### OTHER REFERENCES

. Cangiano C, Laviano A, Del Ben M, et al. Effects of oral 5-hydroxy-tryptophan on energy intake and macronutrient selection in non-insulin dependent diabetic patients. Int J Obes Relat Metab Disord. 1998 July; 22(7):648-54.
2. Cangiano C, Ceci F, Cascino A, et al. Eating behavior and adherence to dietary prescriptions in obese adult subjects treated with 5-hydroxytryptophan. Am J Clin Nutr. 1992 November; 56(5):863-7.
3. Bell S J, Goodrick G K. A functional food product for the management of weight. Crit Rev Food Sci Nutr. 2002 March; 42(2):163-78.
4. Birdsall T C. 5-Hydroxytryptophan: a clinically-effective serotonin precursor. Altern Med Rev. 1998 August; 3(4):271-80.
5. Anon. 5-hydroxytryptophan. Altern Med Rev. 1998 June; 3(3):224-6.
6. DeMyer M K, Shea P A, Hendrie H C, Yoshimura N N. Plasma tryptophan and five other amino acids in depressed and normal subjects. Arch Gen Psychiatry. 1981 June; 38(6):642-6.
7. Ceci F, Cangiano C, Cairella M, et al. The effects of oral 5-hydroxytryptophan administration on feeding behavior in obese adult female subjects. J Neural Transm. 1989; 76(2):109-17.
8. Meijer W C, van Veldhuisen D J, Kema I P, et al. Cardiovascular abnormalities in patients with a carcinoid syndrome. Neth J Med. 2002 March; 60(1):10-6.
9. Meyers S. Use of neurotransmitter precursors for treatment of depression. Altern Med Rev. 2000 February; 5(1):64-71.
10. Hussain A M, Mitra A K. Effect of aging on tryptophan hydroxylase in rat brain: implications on serotonin level. Drug Metab Dispos. 2000 September; 28(9):1038-42.
11. Harrison B J, Olver J S, Norman T R, et al. Selective effects of acute serotonin and catecholamine depletion on memory in healthy women. J Psychopharmacol. 2004 March; 18(1):32-40.
12. Lieberman H R. Nutrition, brain function, and cognitive performance. Appetite. 2003 June; 40(3):245-54.
13. Freeman M P, Helgason C, Hill R A. Selected integrative medicine treatments for depression: considerations for women. J Am Med Womens Assoc. 2004; 59(3):216-24.
14. Poldinger W, Calanchini B, Schwarz W. A functional-dimensional approach to depression: serotonin deficiency as a target syndrome in a comparison of 5-hydroxytryptophan and fluvoxamine. Psychopathology. 1991; 24(2):53-81.
15. Zmilacher K, Battegay R, Gastpar M. L-5-hydroxytryptophan alone and in combination with a peripheral decarboxylase inhibitor in the treatment of depression. Neuropsychobiology. 1988; 20(1):28-35.
16. Juhl J H. Fibromyalgia and the serotonin pathway. Altern Med Rev. 1998 October; 3(5):367-75.
17. Maron E, Toru I, Vasar V, Shlik J. The effect of 5-hydroxytryptophan on cholecystokinin-4-induced panic attacks in healthy volunteers. J Psychopharmacol. 2004 June; 18(2):194-9.
18. Bruni O, Ferri R, Miano S, Verrillo E. L-5-Hydroxytryptophan treatment of sleep terrors in children. Eur J Pediatr. 2004 July; 163(7):402-7.
19. Zajecka J M, Fawcett J. Antidepressant combination and potentiation. Psychiatr Med. 1991; 9(1):55-75.
20. Nardini M, De Stefano R, Iannuccelli M, Borghesi R, Battistini N. Treatment of depression with L-5-hydroxytryptophan combined with chlorimipramine, a double-blind study. Int J Clin Pharmacol Res. 1983; 3(4):239-50.
21. van Praag H, de Hann S. Depression vulnerability and 5-hydroxytryptophan prophylaxis. Psychiatry Res. 1980 September; 3(1):75-83.
22. Das Y T, Bagchi M, Bagchi D, Preuss H G. Safety of 5-hydroxy-L-tryptophan. Toxicol Lett. 2004 Apr. 15; 150(1):111-22.
23. Degner D, Grohmann R, Kropp S, et al. Severe adverse drug reactions of antidepressants: results of the German multicenter drug surveillance program AMSP. Pharmacopsychiatry. 2004 March; 37 Suppl 1S39-S45.
24. Wallace W A, Balsitis M, Harrison B J. Male breast neoplasia in association with selective serotonin re-uptake inhibitor therapy: a report of three cases. Eur J Surg Oncol. 2001 June; 27(4):429-31.
25. Fuller R W, Beasley C M, Jr. Fluoxetine mechanism of action. J Am Acad Child Adolesc Psychiatry. 1991 September; 30(5):849-50.
26. Liebowitz M R, Quitkin F M, Stewart J M, et al. Psychopharmacological dissection of nonendogenous depression. Psychopharmacol Bull. 1984; 20(3):390-2.
27. Thomson J, Rankin H, Ashcroft G W, et al. The treatment of depression in general practice: a comparison of L-tryptophan, amitriptyline, and a combination of L-tryptophan and amitriptyline with placebo. Psychol Med. 1982 November; 12(4):741-51.
28. Marshall J B, Forker A D. Cardiovascular effects of tricyclic antidepressant drugs: therapeutic usage, overdose, and management of complications. Am Heart J. 1982 March; 103(3):401-14.
29. van Praag H M, Uleman A M, Spitz J C. The vital syndrome interview. A structured standard interview for the recognition and registration of the vital depressive symptom complex. Psychiatr Neurol Neurochir. 1965 September; 68(5):32946.
30. Westenberg H G, Gerritsen T W, Meijer B A, van Praag H M. Kinetics of I-5-hydroxytryptophan in healthy subjects. Psychiatry Res. 1982 December; 7(3):373-85.
31. van Praag H M. In search of the mode of action of antidepressants. 5-HTP/tyrosine mixtures in depressions. Neuropharmacology. 1983 March; 22(3 Spec No):433-40.
32. Gelenberg A J, Gibson C J. Tyrosine for the treatment of depression. Nutr Health. 1984; 3(3): 163-73.
33. van Praag H M. In search of the mode of action of antidepressants: 5-HTP/tyrosine mixtures in depression. Adv Biochem Psychopharmacol. 1984; 39:301-14.
34. Attenburrow M J, Williams C, Odontiadis J, et al. The effect of a nutritional source of tryptophan on dieting-induced changes in brain 5-HT function. Psychol Med. 2003 November; 33(8): 1381-6.
35. Yamada J, Sugimoto Y, Ujikawa M. Effects of insulin and adrenalectomy on elevation of serum leptin levels induced by 5-hydroxytryptophan in mice. Biol Pharm Bull. 2003 October; 26(10):1491-3.
36. Itskovitz H D, Werber J L, Sheridan A M, Brewer T F, Stier C T, Jr. 5-Hydroxytryptophan and carbidopa in spontaneously hypertensive rats. J Hypertens. 1989 April; 7(4):311-5.
37. Bertoldi M, Gonsalvi M, Voltattorni C B. Green tea polyphenols: novel irreversible inhibitors of dopa decarboxylase. Biochem Biophys Res Commun. 2001 Jun. 1; 284(1):90-3\\\\
37. U.S. Pat. No. 4,346,410

What is claimed is:

1. Any Pharmaceutical dosage form Nutritional or Dietary supplement, in tablet form enteric or film coated, softgel or hard capsule, injectable, I.V. or other form, containing 1-5000 mg of 5-HTP and 1-5000 mg (EGCG).

2. Same claim as 1 substituting 5-HTP with Levodopa.

3. Same claim as claims 1-2 substituting (EGCG) with (EGC).

4. Same as claims 1-3 with teas or any product containing either of the catechins as a component.

5. Same as claims 1-4 as part of a dietary regimen.

6. Same as claims 1-5 as treatment for depression.

7. Same as claims 1-5 as a weight loss agent.

8. Same as claims 1-5 as a treatment for Fibromyalgia.

9. Same as claims 1-5 as a treatment for Parkinson's Disease or Syndrome.

10. Same as claims 1-5 as a treatment for Myoclonus.

11. Equivalents
Those skilled in the art will recognize, or be able to ascertain using no more than routine experimentation, many equivalents to the specific embodiments of the invention described herein. Such equivalents are intended to be encompassed by the claims contained herein.
